(19) United States
(12) Patent Application Publication
(10) Pub. No.: US 2026/0235569 A1
(43) Pub. Date: Aug. 13, 2026

(54) SENSORS, SYSTEMS, AND METHODS FOR MONITORING PHARMACEUTICAL COMPONENTS

(71) Applicant: West Pharmaceutical Services, Inc., Exton, PA (US)

(72) Inventor: Anthony J. WIRTEL, Malvern, PA (US)

(21) Appl. No.: 19/153,475

(22) PCT Filed: Feb. 5, 2024
(86) PCT No.: PCT/US2024/014495

§ 371 Date: Aug. 4, 2025

Related U.S. Application Data
(60) Provisional application No. 63/443,518, filed on Feb. 6, 2023.

(51) Int. Cl. G01N 33/15

(52) U.S. Cl. CPC G01N 33/15

(57) ABSTRACT

Monitored pharmaceutical components, systems, and methods are described. The monitored pharmaceutical components include a body and electronics within the body. The electronics can detect forces acting on the monitored pharmaceutical component, generate data indicative of the forces over time, and transmit the data. The systems can also include a plurality of unmonitored pharmaceutical components. Each unmonitored pharmaceutical component can include a body that substantially corresponds to the body of the monitored pharmaceutical component. The system can also include a device external from the monitored pharmaceutical component. The device can receive, from the electronics of the monitored pharmaceutical component, the data. The device can predict, based on the data indicative of the forces acting on the monitored pharmaceutical component over time, degradation of the plurality of unmonitored pharmaceutical components.

### CROSS REFERENCE TO RELATED APPLICATION

This application claims priority to U.S. Provisional Patent App. No. 63/443,518, filed on Feb. 6, 2023, the entirety of which is hereby incorporated herein by reference.

### TECHNICAL FIELD

Sensors, systems, and methods for monitoring components, particularly for monitoring the forces experienced by pharmaceutical components used to seal pharmaceutical containers during processing and/or shipping of the pharmaceutical components, are described.

### BACKGROUND

Components, such as stoppers, seals, pistons, plungers, etc., can be formed of polymers (e.g., elastomers), or other similar materials. Polymers and other similar materials can be desirable for forming the components for durability and for material properties that allow for sealing engagement with containers, such as vials or syringes, that can store pharmaceutical substances.

Such components can be subjected to a number of different forces during processing of the components. For example, processing of components can subject the components to forces such as when the components are dropped into an accumulation tank, vibrated in a vibrating bowl, handled by workers or machines, conveyed, pressed into sealing engagement with medical containers, etc. Although forming the components out of polymers or other similar materials can mitigate degradation that can result from experiencing such forces, degradation is not always eliminated by material selection alone. Because different users of components can process components differently and due to variations within the same processes, the forces experienced by components can vary significantly. But such forces experienced by components during processing are neither well quantified nor well understood.

### SUMMARY

There exists an unmet need to better quantify and understand forces that components are subject to during processing and to correlate such forces to degradation of particular components. This need is particularly acute for components that can come into direct contact with substances for human treatment or consumption, to further mitigate or eliminate contamination and/or compromised functionality that can result from excess degradation of the components. These needs are met, to a great extent, by monitoring systems according to some aspects of this invention. The monitoring system includes a monitored pharmaceutical component. The monitored pharmaceutical component can include a body and electronics within the body. The electronics can detect forces acting on the monitored pharmaceutical component, can generate data indicative of the forces acting on the monitored pharmaceutical component over time, and can transmit the data. The monitoring system also include a plurality of unmonitored pharmaceutical components. Each unmonitored pharmaceutical component of the plurality of unmonitored pharmaceutical components can include a body. The body of each unmonitored pharmaceutical component substantially corresponding to the body of the monitored pharmaceutical component. The monitoring system also can include a device external from the monitored pharmaceutical component. The device can include a processor. The device can: receive, from the electronics of the monitored pharmaceutical component, the data indicative of the forces acting on the monitored pharmaceutical component over time; and predict, based on the data indicative of the forces acting on the monitored pharmaceutical component over time, degradation of the plurality of unmonitored pharmaceutical components.

Implementations may include one or more of the following features. The body of the monitored pharmaceutical component has a size, a mass, and a shape, and the body of each unmonitored pharmaceutical component of the plurality of unmonitored pharmaceutical components has a size, a mass, and a shape that substantially correspond to the respective size, mass, and shape of the body of the monitored pharmaceutical component. The body of the monitored pharmaceutical component may include a polymer, and the body of each unmonitored pharmaceutical component of the plurality of unmonitored pharmaceutical components may include the polymer. The polymer is an elastomer. The body of the monitored pharmaceutical component and the body of each of the unmonitored pharmaceutical components is at least one of a stopper, a seal, a piston, or a plunger. The unmonitored pharmaceutical components are configured to seal a pharmaceutical container, and the pharmaceutical container is at least one of a vial or a syringe. The processor of the device is configured to predict the degradation of the plurality of unmonitored pharmaceutical components based on the data indicative of the forces acting on the monitored pharmaceutical component over time and based on historical data for other monitored pharmaceutical components. The historical data is received by the processor before the data indicative of the forces acting on the monitored pharmaceutical component over time. The historical data may include: data indicative of forces acting on bodies of the other monitored pharmaceutical components over time; and data of degradation on the bodies of the other monitored pharmaceutical components. The historical data includes an association of the data indicative of forces acting on the bodies of the other monitored pharmaceutical components over time with the data of degradation on the bodies of the other monitored pharmaceutical components. The association of the data indicative of forces acting on the bodies of the other monitored pharmaceutical components over time with the data of degradation on the bodies of the other monitored pharmaceutical components is a model associating the data indicative of forces acting on the bodies of the other monitored pharmaceutical components over time with the data of degradation on the bodies of the other monitored pharmaceutical components. The model is a linear regression model based on the historical data. The model is a machine learning model trained by the historical data. The processor of the device is configured to predict the degradation of the plurality of unmonitored pharmaceutical components based on the data indicative of the forces acting on the monitored pharmaceutical component over time and the model. The processor of the device is configured to automatically generate an alert based on the predicted degradation of the plurality of unmonitored pharmaceutical components being outside of a threshold. The threshold is a threshold amount of degradation, and the processor of the device is configured to automatically generate the alert based on the predicted degradation of the plurality of unmonitored pharmaceutical components being above the threshold amount of degradation. The threshold is a threshold life remaining of the plurality of unmonitored pharmaceutical components, and the processor of the device is configured to automatically generate the alert based on the predicted degradation of the plurality of unmonitored pharmaceutical components being below the threshold life remaining. The monitoring system may include associating, with the processor of the device, the data indicative of the forces acting on the monitored pharmaceutical component over time with an identifier representing the plurality of unmonitored pharmaceutical components. The body of each unmonitored pharmaceutical component of the plurality of unmonitored pharmaceutical components does not include embedded electronics. The body of the monitored pharmaceutical component has a first color, and the body of at least one of the unmonitored pharmaceutical components of the plurality of unmonitored pharmaceutical components has a second color that is different from the first color. The processor is further configured to: determine that the detected forces acting on the monitored pharmaceutical component are below a threshold, and switch to a power conservation mode based on the determination that the detected forces acting on the monitored pharmaceutical component are below the threshold. The processor is further configured to: determine that the detected forces acting on the monitored pharmaceutical component are at or above the threshold, and operate in a power performance mode based on the determination that the detected forces acting on the monitored pharmaceutical component are at or above the threshold. When the processor is in the power conservation mode, the processor does not generate the data indicative of the forces acting on the monitored pharmaceutical component over time. When the processor is in the performance power mode, the processor generates the data indicative of the forces acting on the monitored pharmaceutical component over time. The electronics may include: a sensor configured to detect the forces acting on the body of the monitored pharmaceutical component; a processor configured to communicate with the sensor and to generate the data indicative of the forces acting on the monitored pharmaceutical component over time; a memory configured to store the data indicative of the forces acting on the monitored pharmaceutical component over time; a transmitter configured to transmit the data indicative of the forces acting on the monitored pharmaceutical component over time; and a power supply configured to power the sensor, the processor, the memory, and the transmitter. The sensor may include at least one of an accelerometer, a gyroscope, or a magnetometer. The electronics further may include at least one of a light emitting source or a sound emitting source. A portion of the body of the monitored pharmaceutical component defines a cutout or thinned region to increase transmission from the light emitting source or the sound emitting source.

Another general aspect includes a method that includes generating, at a monitored pharmaceutical component, data indicative of forces acting on the monitored pharmaceutical component over time during at least one of processing or shipping of the monitored pharmaceutical component together with a plurality of unmonitored pharmaceutical components. The method also includes receiving, at a device external from the monitored pharmaceutical component, the data indicative of forces acting on the monitored pharmaceutical component over time. The method also includes predicting, via the device, and based on the data indicative of forces acting on the monitored pharmaceutical component over time, a degradation of the plurality of the unmonitored pharmaceutical components.

Implementations may include one or more of the following features. The monitored pharmaceutical component may include a body, the body of the monitored pharmaceutical component has a size, a mass, and a shape, and each of the unmonitored pharmaceutical components of the plurality of unmonitored pharmaceutical components may include a body, the body of each unmonitored pharmaceutical component of the plurality of unmonitored pharmaceutical components has a size, a mass, and a shape that substantially correspond to the respective size, mass, and shape of the body of the monitored pharmaceutical component. The at least one of the processing or the shipping includes at least one of: storing the monitored pharmaceutical component together with the plurality of unmonitored pharmaceutical components in an accumulation tank; vibrating the monitored pharmaceutical component together with the plurality of unmonitored pharmaceutical components together in a vibrating bowl; conveying the monitored pharmaceutical component together with the plurality of unmonitored pharmaceutical components on a conveyor; conveying the monitored pharmaceutical component together with the plurality of unmonitored pharmaceutical components on a capping and insertion line; conveying the monitored pharmaceutical component together with the plurality of unmonitored pharmaceutical components on a labeling line; or storing the monitored pharmaceutical component together with the plurality of unmonitored pharmaceutical components on a collection table.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 shows a schematic representation of a monitoring system;

FIG. 2 shows a schematic representation of a monitored pharmaceutical component;

FIG. 3 shows a perspective view of a monitored stopper;

FIG. 4 shows a transparent view of the monitored stopper of FIG. 3; and

FIG. 5 shows a monitoring process.

### DETAILED DESCRIPTION

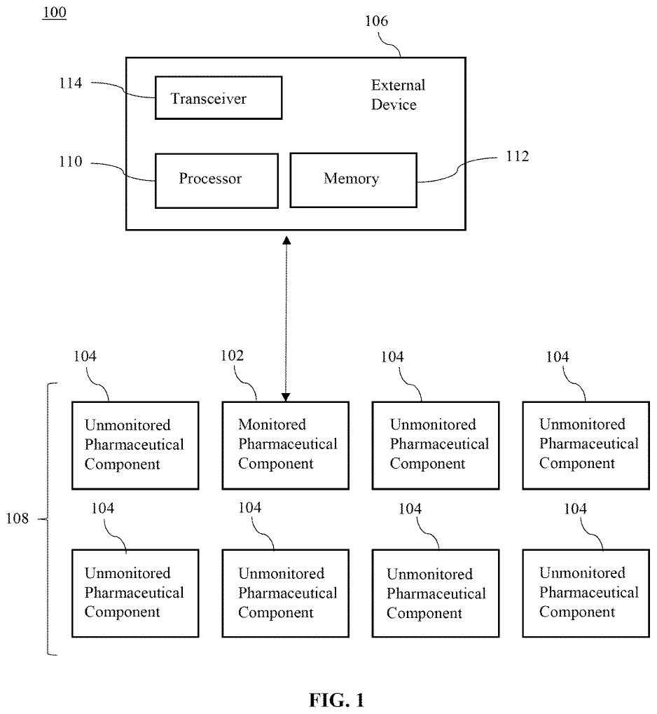

FIG. 1 shows a monitoring system 100 according to aspects of the invention. The monitoring system 100 can include a monitored pharmaceutical component 102 and a plurality of unmonitored pharmaceutical components 104. The monitored pharmaceutical component 102 can include a body with electronics within the body. The electronics can detect forces acting on the monitored pharmaceutical component 102. The electronics can also generate data indicative of the forces acting on the monitored pharmaceutical component 102 and can transmit the data.

Each of the unmonitored pharmaceutical components 104 can include a body. The body of each unmonitored pharmaceutical component 104 can substantially correspond to the body of the monitored pharmaceutical component 102. Because the body of each of the unmonitored pharmaceutical components 104 substantially corresponds to the body of the monitored pharmaceutical component 102, it can be inferred that forces experienced by the monitored pharmaceutical component 102 can be similar to forces experienced by one or more of the unmonitored pharmaceutical components 104 that are processed or shipped together in a batch 108 with the monitored pharmaceutical component 102, as discussed further below. For example, the body of each unmonitored pharmaceutical component 104 can have a size, mass, shape, combinations thereof, among other possibilities, that is substantially similar to (e.g., within +/−10%, within +/−5%, within +/−2.5%, among other possibilities) respective ones of the size, mass, shape, combinations thereof, among other possibilities, of the monitored pharmaceutical component 102. In embodiments, the body of the monitored pharmaceutical component 102 can be formed partially or entirely of the same material (e.g., a polymer such as an elastomer) as the body of each of the unmonitored pharmaceutical components 104. Alternatively, the body of the monitored pharmaceutical component 102 can be formed partially or entirely of a different material than the body of each of the unmonitored pharmaceutical components 104.

In embodiments, the bodies of any or all of the unmonitored pharmaceutical components 104 can be provided without electronics. According to this configuration, the unmonitored pharmaceutical components 104 can be manufactured more easily less expensively while still serving the intended function of sealing the container that can hold the pharmaceutical component. Since the body of the monitored pharmaceutical component 102 can be structurally representative of any or all of the bodies of the unmonitored pharmaceutical components 104, data collected from the monitored pharmaceutical component 102 can be representative of any or all of the unmonitored pharmaceutical components 104. This can make electronics unnecessary within any or all of the unmonitored pharmaceutical components 104 since data collected from the monitored pharmaceutical component 102 during processing can be representative of any or all of the unmonitored pharmaceutical components 104 as well. Alternatively, in some embodiments any or all of the unmonitored pharmaceutical components 104 can include electronics that serve a different function from the electronics encapsulated within the monitored pharmaceutical component 102. For example, any or all of the unmonitored pharmaceutical components 104 can be smart devices that function together with other devices for different purposes than the electronics encapsulated within monitored pharmaceutical component 102. The term “unmonitored pharmaceutical components” in this disclosure can mean either components without any embedded electronics or with embedded electronics that are not configured to monitor the components during processing in the manner as the described monitored pharmaceutical component 102.

In embodiments, the body of the monitored pharmaceutical component 102 and the body of each of the unmonitored pharmaceutical components 104 can be a stopper, a seal, piston, plunger, combinations thereof, among other possibilities. The unmonitored pharmaceutical components 104 can seal a pharmaceutical container such as for example a vial, a syringe, among other possibilities. In embodiments, the monitored pharmaceutical component 102 can seal the pharmaceutical container.

In embodiments the body of the monitored pharmaceutical component 102 can have a different color from a color of any or all of the unmonitored pharmaceutical components 104 such that the monitored pharmaceutical component 102 can be distinguished from any or all of the unmonitored pharmaceutical components 104 without compromising the structural relationship between the body of the monitored pharmaceutical component 102 and the bodies of any or all of the unmonitored pharmaceutical components 104. In additional or alternative embodiments, the monitored pharmaceutical component 102 can include light emitting sources and/or sound emitting sources that can distinguish the monitored pharmaceutical component 102 from any or all of the unmonitored pharmaceutical components 104 without compromising the structural relationship between the body of the monitored pharmaceutical component 102 and the bodies of any or all of the unmonitored pharmaceutical components 104.

The monitoring system 100 can include a device 106 external from the monitored pharmaceutical component 102. The device 106 can include a processor 110. The device 106 can receive, from the electronics of the monitored pharmaceutical component 102, the data indicative of the forces acting on the monitored pharmaceutical component 102 over time. The device 106 can include a memory 112 that can store the data indicative of the forces acting on the monitored pharmaceutical component 102 over time. The device 106 can include a transceiver 114 or a receiver that can receive the data from the monitored pharmaceutical component 102. In embodiments, the transceiver 114 can transmit information from the device 106 to, for example, the monitored pharmaceutical component 102. For example, the processor 110 can control the transceiver 114 to transmit instructions to the monitored pharmaceutical component 102 including for example instructions to start and/or stop generation of the data described herein.

The processor 110 of the device 106 can predict, based on the data indicative of the forces acting on the monitored pharmaceutical component 102 over time, degradation of the plurality of unmonitored pharmaceutical components 104. For example, the monitored pharmaceutical component 102 and the plurality of unmonitored pharmaceutical components 104 can be processed and/or shipped together in the batch 108. The monitored pharmaceutical component 102 can detect forces acting on the monitored pharmaceutical component 102 during the processing and/or shipping of the batch 108 and can generate data indicative of the forces acting on the monitored pharmaceutical component 102 over time (e.g. during the processing or shipping). The device 106 can receive, from the electronics of the monitored pharmaceutical component 102, the data indicative of the forces acting on the monitored pharmaceutical component 102 over time. The device 106 can predict, based on the data indicative of the forces acting on the monitored pharmaceutical component 102 over time, degradation of the plurality of unmonitored pharmaceutical components 104. The predicted degradation can be for each of the unmonitored pharmaceutical components 104 of the plurality of unmonitored pharmaceutical components 104, which can be advantageous to ensure the quality of each of the unmonitored pharmaceutical components 104 within the batch 108. The term “predict” as used herein can include the plain and ordinary meaning and can mean to estimate as a consequence of something. For example, predicting degradation of the unmonitored pharmaceutical components 104 based on the data indicative of the forces acting on the monitored pharmaceutical component 102 over time can mean estimating degradation of the unmonitored pharmaceutical components 104 as a consequence of the forces acting on the monitored pharmaceutical component 102 over time. The device 106 can make the prediction by inferring that the forces acting upon the body of the monitored pharmaceutical component 102 are representative of the forces acting upon the bodies of the unmonitored pharmaceutical components 104 since the bodies substantially correspond to each other and since the bodies are processed and/or shipped together in the batch 108. The predictions of degradation of the unmonitored pharmaceutical components 104 can be advantageous for quantifying and understanding degradation of the unmonitored pharmaceutical components 104, which is particularly useful since they can come into direct contact with substances for human treatment or consumption. The predictions of degradation can help to mitigate or eliminate contamination and/or compromised functionality that can result from excess degradation of such components.

The processor 110 can predict a degradation of the unmonitored pharmaceutical components 104 based upon the data indicative of the forces acting on the monitored pharmaceutical component 102 over time in a number of different or complimentary ways. For example, the processor 110 can predict the degradation of the plurality of unmonitored pharmaceutical components 104 processed and/or shipped together in the batch 108 with the monitored pharmaceutical component 102 based on the data indicative of the forces acting on the monitored pharmaceutical component 102 over time (e.g., during the processing and/or shipping) and based on historical data for other monitored pharmaceutical components 102 than can serve as reference monitored pharmaceutical components. The other monitored pharmaceutical components 102 can be identical to the monitored pharmaceutical component 102, but not part of the batch 108. The historical data can be received by the processor 110 before receipt of the data indicative of the forces acting on the monitored pharmaceutical component 102 over time. The historical data can include data indicative of forces acting on bodies of the other monitored pharmaceutical components 102 over time, and data of degradation on the bodies of the other monitored pharmaceutical components 102. The data of degradation on the bodies of the other monitored pharmaceutical components 102 can qualitative (e.g., based on the judgement of a human observer) or quantitative (measured using any number of known techniques for measuring degradation including 3D scanning, physical measurements, weight differences before and after the degradation, combinations thereof, among other possibilities). The historical data can include an association of the data indicative of the forces acting on the bodies of the other monitored pharmaceutical components 102 over time with the data of degradation on the bodies of the other monitored pharmaceutical components 102. The association of the data indicative of forces acting on the bodies of the other monitored pharmaceutical components 102 over time with the data of degradation on the bodies of the other monitored pharmaceutical components can be a model that associates the data indicative of forces acting on the bodies of the other monitored pharmaceutical components 102 over time with the data of degradation on the bodies of the other monitored pharmaceutical components 102. The model can be a linear regression model based on the historical data, a machine learning model trained by the historical data, combinations thereof, among other possibilities. In embodiments, the processor 110 can predict a degradation of the plurality of unmonitored pharmaceutical components 104 based on the data indicative of the forces acting on the monitored pharmaceutical component over time and the model.

In embodiments, the processor 110 can automatically generate an alert based on the prediction of degradation of the plurality of unmonitored pharmaceutical components 104 being outside of the threshold. The alert can be visual such as a pop-up screen on a display of the device 106, auditory such as a sound output from a speaker of the device 106, combinations thereof, among other possibilities. The threshold can be a threshold amount of degradation and the processor 110 can automatically generate the alert based on a prediction of degradation of the plurality of unmonitored pharmaceutical components 104 being above the threshold amount of degradation. Additionally or alternatively, the threshold can be a threshold life remaining of the plurality of unmonitored pharmaceutical components 104. In such embodiments, the processor 110 can automatically generate the alert based on a prediction of degradation of the plurality of unmonitored pharmaceutical components 104 being below the threshold life remaining.

In embodiments, the processor 110 can associate the data indicative of the forces acting on the monitored pharmaceutical component 102 over time with an identifier representing the plurality of unmonitored pharmaceutical components 104 within the batch 108. The identifier can be any unique identifying feature (e.g., such as a serial number) that can represent the batch 108 as a whole. The identifier can be advantageous for tracking the history of forces experienced by the batch 108.

In embodiments, the electronics (e.g., a processor of the electronics) of the monitored component 104 can operate in either a power performance mode or a power conservation mode that consumes less power than the power performance mode. For example, the processor of the electronics of the monitored component 104 can default to the power performance mode and can generate the data indicative of the forces acting on the monitored pharmaceutical component 102 over time and store the data indicative of the forces acting on the monitored pharmaceutical component 102 over time (e.g., in a memory of the electronics) when in the power performance mode. The processor of the electronics of the monitored component 104 can determine that the forces acting on the body of the monitored pharmaceutical component 102 detected by the electronics are below the threshold and based on that determination can switch to the power conservation mode. When in the power conservation mode, the processor of the electronics of the monitored component 104 can not generate the data indicative of the forces acting on the monitored pharmaceutical component 102 over time. This can be advantageous in that the processor 110 can consume less power by not recording forces below the threshold, such forces being less likely to cause degradation than forces above the threshold. The processor of the electronics of the monitored component 104 can also determine that the forces acting on the body of the monitored pharmaceutical component 102 detected by the electronics are at or above the threshold and switch back to power performance mode based on that determination.

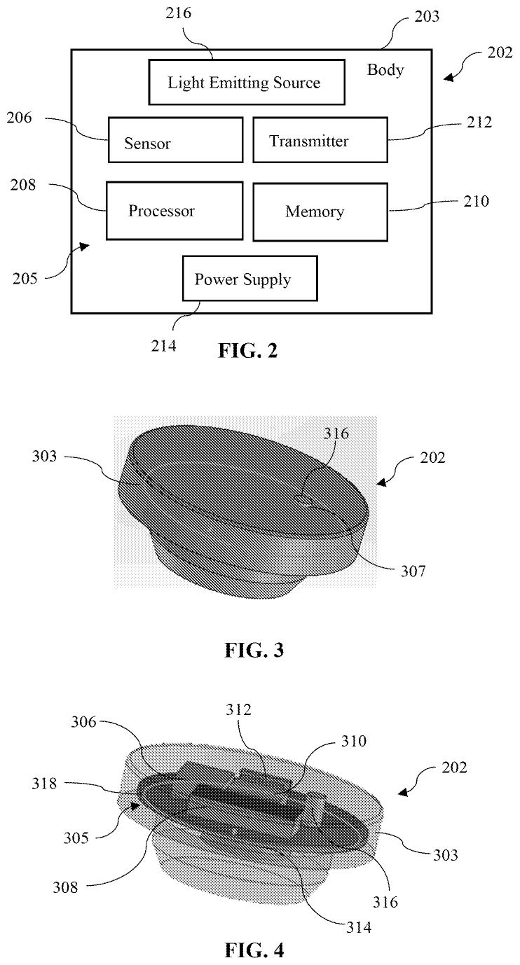

FIG. 2 shows a schematic view of an embodiment of the monitored pharmaceutical component 202 according to aspects of the invention. The monitored pharmaceutical component 202 can include any or each of the structures, features, relationships, etc. previously described with respect to the monitored pharmaceutical component 102, and vice versa. For example, the monitored pharmaceutical component 202 can be used in the monitoring system 100 in any or all of the manners previously described with respect to the monitored pharmaceutical component 102. The monitored pharmaceutical component 202 can include the body 203 and the electronics 205. The electronics 205 can be partially or completely encased and/or encapsulated within the body 203. For example, the body 203 can be molded around the electronics 205.

The body 203 can be formed of a polymer (e.g., an elastomer) or other similar material. The body 203 can be sized and shaped to seal a corresponding container that can store a pharmaceutical substance. For example, in embodiments the body 203 can form a stopper, seal, piston, plunger, or other component that is complementary to an associated container, such as a vial or syringe, for operative connection with the associated container.

The electronics 205 can include a sensor 206. The sensor 206 can detect forces acting on the body 203. In embodiments, the sensor 206 can include at least one of an accelerometer, a gyroscope, or a magnetometer. In embodiments, the sensor 206 can include a three-axis accelerometer with a +/−16 g minimum measurement range, though other ranges are possible.

The electronics 205 can include a processor 208 and a memory 210. The processor 208 can be operatively connected to the sensor 206. The processor 208 can receive data of, for example, the forces acting on the body 203 from the sensor 206. Although force data is described throughout this disclosure, in embodiments the processor 208 can receive data from any of the sensors 206 described herein and can transmit any other data for use in determining information regarding the monitored pharmaceutical component 202 during processing. Upon receipt of the data of the forces acting on the body 203 from the sensor 206, the processor 208 can automatically generate the data indicative of the forces acting on the monitored pharmaceutical component 202 over time. The memory 210 can store the data indicative of the forces acting on the monitored pharmaceutical component 202 over time. This can be advantageous in that it can enable intermittent transmission of the data to the device 106 without loss of data.

In embodiments, the electronics 205 can include a transmitter 212. The transmitter 212 can be operatively connected to the processor 208 and can be controlled by the processor 208. For example, in embodiments the processor 208 can automatically control the transmitter 212 to transmit the data from the monitored pharmaceutical component 202 to another device, such as for example the device 106. The data transmitted from the monitored pharmaceutical component 202 can be used by the other device to predict degradation, as previously described. Alternatively, the electronics 205 can be provided without a transmitter 212. In such embodiments, the data can be extracted from the memory 210 via direct connection with the electronics 205 (which my require destruction of the body 203).

The electronics 205 can include a power supply 214. The power supply 214 can power the sensor 206, the processor 208, the memory 210, the transmitter 212, and/or any other aspect of the electronics 205. In embodiments, the power supply 214 can include a battery or functionally similar structure.

In embodiments, the electronics 205 can include a light emitting source 216 and/or a sound emitting source. The light emitting source 216 and/or sound emitting source can help to distinguish the monitored pharmaceutical component 202 from other similar components and make the monitored pharmaceutical component 202 easier to track. In embodiments, a portion of the body 203 can define a cutout or thinned region to increase transmission of light or sound from the light emitting source 216 or the sound emitting source. The light emitting source 216 and/or the sound emitting source can be powered by the power supply 214. In embodiments, the light emitting source can emit visible wavelengths of light and/or ultraviolet and/or infrared and/or any other wavelength of light. In embodiments, the light emitting source 216 can be LED, OLED, incandescent, or other functionally similar structure. The sound emitting source can a speaker or other functionally similar structure. The sound emitting source can emit sounds audible to humans with average hearing and/or sounds that are inaudible to humans with average hearing but that can be detected by machines. In embodiments, the processor 208 can control the light emitting source and/or the sound emitting source for communication purposes. For example, the processor 208 can control the light emitting source to flash to communicate location information, to indicate that a threshold force has been reached, among other possibilities.

In embodiments, the monitored pharmaceutical component 202 can be rechargeable and/or reusable.

FIGS. 3 and 4 show views of a monitored stopper 302 according to some aspects of the invention. The monitored stopper 302 can include any or each of the structures, features, relationships, etc. previously described with respect to the monitored pharmaceutical components 102, 202, and vice versa. For example, the monitored stopper 302 can include a body 303 with electronics 305 therein. The electronics 305 can include a sensor 306, a processor 308, a memory 310, a transmitter 312, a power supply 314, and a light emitting source 316. Any or all of the sensor 306, processor 308, memory 310, transmitter 312, power supply 314, or light emitting source 316 can be a part of a printed circuit board 318. In embodiments, a portion of the body 303 can define the cutout 307 or thinned region for transmitting light from the light emitting source 316. The stopper 302 can seal a vial or syringe that can contain a pharmaceutical substance.

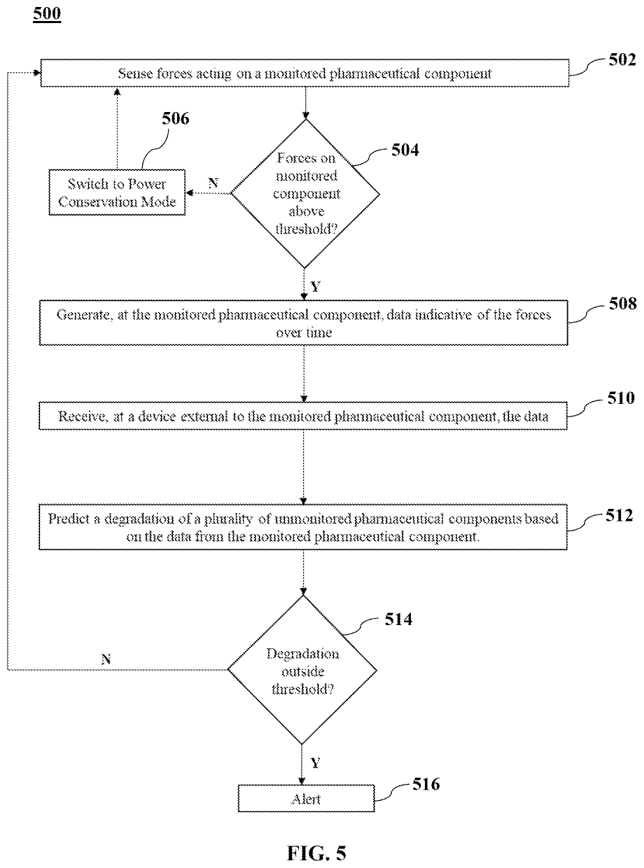

FIG. 5 shows a process 500 of monitoring according to some aspects of the invention. The process 500 can be performed with any of the previously described monitored pharmaceutical components 102, 202, 302, and/or the monitoring system 100.

The process 500 can include, at step 502, sensing forces acting on a monitored pharmaceutical component, as previously described. The sensing of the forces acting on the monitored pharmaceutical component can be performed concurrently with processing and/or shipping the monitored pharmaceutical component together with a plurality of unmonitored pharmaceutical components (e.g., unmonitored pharmaceutical components 104) in a batch, as previously described. Processing and/or shipping the monitored pharmaceutical component together with a plurality of unmonitored pharmaceutical components in the batch can include any or all of: storing the plurality of unmonitored pharmaceutical components together with the monitored pharmaceutical component in an accumulation tank; vibrating the plurality of unmonitored pharmaceutical components together with the monitored pharmaceutical component in a vibrating bowl; conveying the plurality of unmonitored pharmaceutical components together with the monitored pharmaceutical component on a conveyor; conveying the plurality of unmonitored pharmaceutical components together with the monitored pharmaceutical component on a capping and insertion line; conveying the plurality of unmonitored pharmaceutical components together with the monitored pharmaceutical component on a labeling line; storing the plurality of unmonitored pharmaceutical components together with the monitored pharmaceutical component on a collection table, shipping the plurality of unmonitored pharmaceutical components together with the monitored pharmaceutical component, combinations thereof, among other possibilities. In embodiments, any or all of the steps of the process 500 described herein can occur during the processing and/or shipping.

The process 500 can include, at step 504, comparing the forces on the monitored pharmaceutical component to a threshold. For example, the electronics of the monitored pharmaceutical component can compare the sensed forces to a threshold force. If the sensed forces are less than the threshold force, at step 506 of the process 500 the electronics of the monitored pharmaceutical component can switch from a default power performance mode (previously described) to a power conservation mode (previously described). In embodiments, the threshold force can be set by a user to correspond to a magnitude of force that is of sufficient interest to the user. Alternatively, the threshold force can be automatically set (e.g., the processor 110) based on historical data, such as the previously described historical data. In embodiments, the process 500 can repeat steps 502-506 until the sensed forces exceed the threshold force. In embodiments, the process 500 can be performed without steps 504 and 506. In such embodiments, the process 500 can proceed directly from step 502 to step 508.

If the sensed force is greater than or equal to the threshold force at step 504, the process 500 can proceed to step 508 at which the processor of the monitored pharmaceutical component can generate the data indicative of the forces acting on the body of the monitored pharmaceutical component over time, as previously described. If in the power conservation mode, the electronics can switch back to the default power performance mode based on determining that the sensed force is greater than the threshold force at step 504 and before generating the data at step 508.

The process 500 can include, at step 510, receiving, at a processor of a device (e.g., the processor 110 of the device 106) the data generated at step 508.

The process 500 can include, at step 512, predicting, via the device, and based on the data indicative of forces acting on the monitored pharmaceutical component over time, a degradation of the plurality of the unmonitored pharmaceutical components, as previously described.

In embodiments, the processor can include at step 514 comparing the degradation predicted at step 512 to a threshold degradation. The threshold degradation can be set by a user and can correspond to an unacceptable magnitude of degradation and/or a lifetime of the pharmaceutical component(s), as previously described. If the degradation predicted at step 512 is outside of the threshold degradation (as previously described), the process 500 can proceed to step 516 in which an alert can be triggered, as previously described. In response to receiving the alert a user can initiate an investigation of the unmonitored pharmaceutical components processed and/or shipped together with the monitored pharmaceutical component in the batch to determine whether the unmonitored pharmaceutical components need to be discarded or can be used for their intended purpose.

In embodiments, if the degradation predicted at step 512 is not outside of the threshold degradation the process 500 can repeat steps 502-514 for as long as the processing and/or shipping continues. Alternatively, if the processing and/or shipping has completed and the degradation predicted at step 512 is not outside of the threshold degradation, the process 500 can end. In embodiments, the process 500 can be performed without steps 514 and 516.

The term “processor” used throughout this disclosure can include structures in accordance with the plain and ordinary meaning of the term and can include, for example, computing devices with one or more processors coupled to a system memory via an input/output (I/O) interface.

The terms “transmitter” and/or “transceiver” used throughout this disclosure can include structures in accordance with the plain and ordinary meaning of such terms and/or can include any network interface that can allow data to be exchanged (wirelessly or via wired connections) between processors, other devices, networks, etc. Such other devices, such as the device 106, can include displays and/or other user interfaces to allow users to interact with the processors of this disclosure.

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

1. A monitoring system comprising:
a monitored pharmaceutical component, the monitored pharmaceutical component comprising:
a body;
electronics within the body, the electronics being configured to detect forces acting on the monitored pharmaceutical component, to generate data indicative of the forces acting on the monitored pharmaceutical component over time, and to transmit the data;
a plurality of unmonitored pharmaceutical components, each unmonitored pharmaceutical component of the plurality of unmonitored pharmaceutical components comprising a body, the body of each unmonitored pharmaceutical component substantially corresponding to the body of the monitored pharmaceutical component; and
a device external from the monitored pharmaceutical component, the device comprising a processor, and the device being configured to:
receive, from the electronics of the monitored pharmaceutical component, the data indicative of the forces acting on the monitored pharmaceutical component over time, and
predict, based on the data indicative of the forces acting on the monitored pharmaceutical component over time, degradation of the plurality of unmonitored pharmaceutical components.

2. The monitoring system of claim 1, wherein:
the body of the monitored pharmaceutical component has a size, a mass, and a shape, and
the body of each unmonitored pharmaceutical component of the plurality of unmonitored pharmaceutical components has a size, a mass, and a shape that substantially correspond to the respective size, mass, and shape of the body of the monitored pharmaceutical component.

3-4. (canceled)

5. The monitoring system of claim 1, wherein the body of the monitored pharmaceutical component and the body of each of the unmonitored pharmaceutical components is at least one of a stopper, a seal, a piston, or a plunger.

6. The monitoring system of claim 1, wherein:
the unmonitored pharmaceutical components are configured to seal a pharmaceutical container, and
the pharmaceutical container is at least one of a vial or a syringe.

7. The monitoring system of claim 1, wherein the processor of the device is configured to predict the degradation of the plurality of unmonitored pharmaceutical components based on the data indicative of the forces acting on the monitored pharmaceutical component over time and based on historical data for other monitored pharmaceutical components.

8. The monitoring system of claim 7, wherein:
the historical data is received by the processor before the data indicative of the forces acting on the monitored pharmaceutical component over time, and
the historical data comprises:
data indicative of forces acting on bodies of the other monitored pharmaceutical components over time; and
data of degradation on the bodies of the other monitored pharmaceutical components.

9. The monitoring system of claim 8, wherein the historical data includes an association of the data indicative of forces acting on the bodies of the other monitored pharmaceutical components over time with the data of degradation on the bodies of the other monitored pharmaceutical components.

10. The monitoring system of claim 9, wherein the association of the data indicative of forces acting on the bodies of the other monitored pharmaceutical components over time with the data of degradation on the bodies of the other monitored pharmaceutical components is a model associating the data indicative of forces acting on the bodies of the other monitored pharmaceutical components over time with the data of degradation on the bodies of the other monitored pharmaceutical components.

11. The monitoring system of claim 10, wherein the model is a linear regression model based on the historical data or a machine learning model trained by the historical data.

12-13. (canceled)

14. The monitoring system of claim 7, wherein the processor of the device is configured to automatically generate an alert based on the predicted degradation of the plurality of unmonitored pharmaceutical components being outside of a threshold.

15. The monitoring system of claim 14, wherein:
the threshold is a threshold amount of degradation, and
the processor of the device is configured to automatically generate the alert based on the predicted degradation of the plurality of unmonitored pharmaceutical components being above the threshold amount of degradation.

16. The monitoring system of claim 14, wherein:
the threshold is a threshold life remaining of the plurality of unmonitored pharmaceutical components, and
the processor of the device is configured to automatically generate the alert based on the predicted degradation of the plurality of unmonitored pharmaceutical components being below the threshold life remaining.

17. (canceled)

18. The monitoring system of claim 1, wherein the body of each unmonitored pharmaceutical component of the plurality of unmonitored pharmaceutical components does not include embedded electronics.

19. (canceled)

20. The monitoring system of claim 1, wherein the processor is further configured to:
determine that the detected forces acting on the monitored pharmaceutical component are below a threshold,
switch to a power conservation mode based on the determination that the detected forces acting on the monitored pharmaceutical component are below the threshold.
determine that the detected forces acting on the monitored pharmaceutical component are at or above the threshold, and
operate in a power performance mode based on the determination that the detected forces acting on the monitored pharmaceutical component are at or above the threshold.

21. (canceled)

22. The monitoring system of claim 20, wherein:
when the processor is in the power conservation mode, the processor does not generate the data indicative of the forces acting on the monitored pharmaceutical component over time, and
when the processor is in the performance power mode, the processor generates the data indicative of the forces acting on the monitored pharmaceutical component over time.

23. The monitoring system of claim 1, wherein the electronics comprise:
a sensor configured to detect the forces acting on the body of the monitored pharmaceutical component;
a memory configured to store the data indicative of the forces acting on the monitored pharmaceutical component over time;
a transmitter configured to transmit the data indicative of the forces acting on the monitored pharmaceutical component over time; and
a power supply configured to power the sensor, the processor, the memory, and the transmitter.

24. The monitoring system of claim 23, wherein the sensor comprises at least one of an accelerometer, a gyroscope, or a magnetometer.

25-26. (canceled)

27. A method comprising:
generating, at a monitored pharmaceutical component, data indicative of forces acting on the monitored pharmaceutical component over time during at least one of processing or shipping of the monitored pharmaceutical component together with a plurality of unmonitored pharmaceutical components,
receiving, at a device external from the monitored pharmaceutical component, the data indicative of forces acting on the monitored pharmaceutical component over time; and
predicting, via the device, and based on the data indicative of forces acting on the monitored pharmaceutical component over time, a degradation of the plurality of the unmonitored pharmaceutical components.

28. The method of claim 27, wherein:
the monitored pharmaceutical component comprises a body, the body of the monitored pharmaceutical component has a size, a mass, and a shape, and
each of the unmonitored pharmaceutical components of the plurality of unmonitored pharmaceutical components comprises a body, the body of each unmonitored pharmaceutical component of the plurality of unmonitored pharmaceutical components has a size, a mass, and a shape that substantially correspond to the respective size, mass, and shape of the body of the monitored pharmaceutical component.

29. The method of claim 27, wherein the at least one of the processing or the shipping includes at least one of:
storing the monitored pharmaceutical component together with the plurality of unmonitored pharmaceutical components in an accumulation tank;
vibrating the monitored pharmaceutical component together with the plurality of unmonitored pharmaceutical components together in a vibrating bowl;
conveying the monitored pharmaceutical component together with the plurality of unmonitored pharmaceutical components on a conveyor,
conveying the monitored pharmaceutical component together with the plurality of unmonitored pharmaceutical components on a capping and insertion line;
conveying the monitored pharmaceutical component together with the plurality of unmonitored pharmaceutical components on a labeling line; or
storing the monitored pharmaceutical component together with the plurality of unmonitored pharmaceutical components on a collection table.
